(19) United States
(12) Patent Application Publication
(10) Pub. No.: US 2024/0156752 A1
(43) Pub. Date: May 16, 2024

(54) ENDOXIFEN FOR TREATMENT OF OVARIAN CANCER

(71) Applicant: Atossa Therapeutics, Inc., Seattle, WA (US)

(72) Inventor: Steven C. QUAY, Seattle, WA (US)

(21) Appl. No.: 18/279,577

(22) PCT Filed: Mar. 10, 2022
(86) PCT No.: PCT/US2022/019674

§ 371 Date: Aug. 30, 2023

Related U.S. Application Data
(60) Provisional application No. 63/159,548, filed on Mar. 11, 2021.

(51) Int. Cl. A61K 31/138; A61K 9/20; A61K 31/4439; A61P 35/00

(52) U.S. Cl. CPC A61K 31/138; A61K 9/20; A61K 31/4439; A61P 35/00

(57) ABSTRACT

Described herein are compositions and methods for treating ovarian cancer. A method of treating ovarian cancer may include administering endoxifen in combination with a phosphoinositide 3-kinase inhibitor, such as alpelisib.

### CROSS-REFERENCE

The present application claims the benefit of U.S. Provisional Application No. 63/159,548, entitled “ENDOXIFEN FOR TREATMENT OF OVARIAN CANCER,” filed on Mar. 11, 2021, which application is herein incorporated by reference in its entirety for all purposes.

### BACKGROUND

Ovarian cancer is the fifth leading cancer in women, resulting in more than 21,000 cases and more than 13,000 deaths per year in the United States. It is the most common cancer found in female reproductive organs. There is a need for improved methods of treating ovarian cancer.

### SUMMARY

In various aspects, the present disclosure provides a method of treating ovarian cancer in a subject in need thereof, the method comprising administering to the subject a therapeutically effective amount of endoxifen and a phosphoinositide 3-kinase inhibitor, thereby treating the ovarian cancer.

In certain aspects, the endoxifen is Z-endoxifen. In certain aspects, the phosphoinositide 3-kinase inhibitor is selected from the group consisting of alpelisib, idelalisib, copanlisib, duvelisib, umbralisib, taselisib, perifosine, buparlisib, dactolisib, and voxtalisib. In certain aspects, the phosphoinositide 3-kinase inhibitor is alpelisib.

In certain aspects, the endoxifen is administered orally, intravenously, intra-arterially, intra-ovarianly, vaginally, parenterally, topically, or transdermally. In certain aspects, the phosphoinositide 3-kinase inhibitor is administered orally, intravenously, intra-arterially, intra-ovarianly, vaginally, parenterally, topically, or transdermally. In certain aspects, the endoxifen, the phosphoinositide 3-kinase inhibitor, or both are administered orally.

In certain aspects, the endoxifen is formulated as a sustained-release composition. In certain aspects, the endoxifen is formulated as a delayed-release composition. In certain aspects, the endoxifen is administered to the subject daily. In certain aspects, the endoxifen is administered to the subject at a dose of no less than 1 mg and no more than 160 mg per day. In certain aspects, the endoxifen is administered to the subject at a dose of no less than 1 mg and no more than 40 mg per day. In certain aspects, the endoxifen is administered to the subject at a dose of no less than 1 mg and no more than 10 mg per day. In certain aspects, the endoxifen is administered to the subject at a dose of no less than 2 mg and no more than 5 mg per day.

In certain aspects, the phosphoinositide 3-kinase inhibitor is administered to the subject daily. In certain aspects, the phosphoinositide 3-kinase inhibitor is administered to the subject at a dose of no less than 10 mg and no more than 600 mg per day. In certain aspects, the phosphoinositide 3-kinase inhibitor is administered to the subject at a dose of no less than 50 mg and no more than 400 mg per day. In certain aspects, the phosphoinositide 3-kinase inhibitor is administered to the subject at a dose of no less than 150 mg and no more than 300 mg per day.

In certain aspects, the endoxifen is administered 1, 2, 3, or 4 times per day. In certain aspects, the phosphoinositide 3-kinase inhibitor is administered 1, 2, 3, or 4 times per day. In certain aspects, the endoxifen and the phosphoinositide 3-kinase inhibitor are administered once per day. In certain aspects, the endoxifen, the phosphoinositide 3-kinase inhibitor, or both are administered for at least 7, at least 14, at least 21, or at least 28 days. In certain aspects, the endoxifen and the phosphoinositide 3-kinase inhibitor are administered as a single tablet.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The present disclosure provides methods of treating ovarian cancer using endoxifen. Endoxifen is an active metabolite of tamoxifen. In the liver, is broken down into active compounds, or metabolites. One of the active tamoxifen metabolites is endoxifen. (Z)-endoxifen has robust antitumor and anti-estrogenic activity compared to tamoxifen therapy and aromatase inhibitor therapy. Endoxifen (e.g., (Z)-endoxifen) is a selective estrogen receptor modulator (SERM) that functions as a competitive partial agonist of estrogen receptor in a tissue-specific manner. Estrogen receptor is over-expressed in about 70% of breast cancers.

A method of treating ovarian cancer may comprise administering an endoxifen composition to a subject. The endoxifen composition may comprise (Z)-endoxifen, and polymorphs and salts thereof, and methods of making them. In some embodiments, the method may further comprise administering an inhibitor of phosphoinositide 3-kinase (PI3K). For example, the method may comprise administering alpelisib, idelalisib, copanlisib, duvelisib, umbralisib, taselisib, perifosine, buparlisib, dactolisib, or voxtalisib. In some embodiments, the PI3K inhibitor may be an α-specific PI3K inhibitor, such as alpelisib. An example of a method of treating ovarian cancer comprises administering (Z)-endoxifen in combination with alpelisib.

Phosphoinositide 3-kinase is a family of lipid kinases that are frequently mutated to have increased activity in cancer. An alpha subunit of PI3K, PIK3CA (p110α), is mutated or amplified in 30.5% of all ovarian cancers and 45% of the endometrioid and clear cell subtypes of ovarian cancers.

### Methods of Treating Ovarian Cancer

A method of treating ovarian cancer may comprise administering to a subject a combination therapy comprising endoxifen (e.g., (Z)-endoxifen) and a phosphoinositide 3-kinase inhibitor (e.g., alpelisib). In some embodiments, the endoxifen, the phosphoinositide 3-kinase inhibitor, or both are administered orally. In some embodiments, the endoxifen, the phosphoinositide 3-kinase inhibitor, or both are administered intravenously, intra-arterially, intra-ovarianly, vaginally, parenterally, topically, or transdermally. In some embodiments, the endoxifen, the phosphoinositide 3-kinase inhibitor, or both are formulated as a sustained release composition.

A combination therapy comprising endoxifen and a phosphoinositide 3-kinase inhibitor may be administered 1, 2, 3, 4, 5, 6, 7, 8, 9, or 10 times per day. In some embodiments, a composition comprising endoxifen may be administered 1, 2, 3, 4, 5, 6, 7, 8, 9, or 10 times per day. In some embodiments, a composition comprising a phosphoinositide 3-kinase inhibitor may be administered 1, 2, 3, 4, 5, 6, 7, 8, 9, or 10 times per day. Treatment may be administered for at least 7, 14, 21, 28, 30, 35, 42, 49, 56, or 60 days. In some embodiments, treatment may be administered until an ovarian cancer is treated (e.g., by eliminating or reducing the size of a tumor). In some embodiments, treatment may be administered until unacceptable toxicity is observed in the subject.

In some embodiments, endoxifen (e.g., (Z)-endoxifen) may be administered to treat ovarian cancer at a dose of from about 1 mg to about 160 mg, from about 1 mg to about 40 mg, from about 1 mg to about 10 mg, or from about 2 mg to about 5 mg per day. In some embodiments, a phosphoinositide 3-kinase inhibitor (e.g., alpelisib) may be administered to treat ovarian cancer at a dose of from about 10 mg to about 600 mg, from about 50 mg to about 400 mg, or from about 150 mg to about 300 mg per day.

An example of a method of treating ovarian cancer in a subject comprises orally administering to the subject 4 mg of (Z)-endoxifen per day and 300 mg of alpelisib per day. The (Z)-endoxifen and alpelisib may be administered in combination daily for at least 28 days, or until tumor size decreases, or until unacceptable toxicity is observed.

### Endoxifen Formulations

A method of treating ovarian cancer may comprise administering a composition comprising endoxifen to the subject.

As used herein, “endoxifen” may refer to compositions comprising the (Z)-isoform, referred to as “(Z)-endoxifen,” the (E)-isoform, referred to as “(E)-endoxifen,” or a mixture of (Z)-isoform and (E)-isoform, referred to as “(E/Z)-endoxifen.” The present disclosure provides compositions comprising (Z)-endoxifen. In some embodiments, the (Z)-endoxifen may comprise a stable (Z)-endoxifen free base, or polymorphs or salts thereof. In some embodiments, the compositions may further comprise (E)-endoxifen. In some embodiments, the compositions may comprise endoxifen predominantly in the form of (Z)-endoxifen free base. Unless specifically referred to by the prefix (Z), (E) or (E/Z), endoxifen used generally without a prefix is used herein to include to any or all endoxifen isoforms.

A mixture of (E)-endoxifen and (Z)-endoxifen, also referred to as (E/Z)-endoxifen, can be represented by Formula (I):

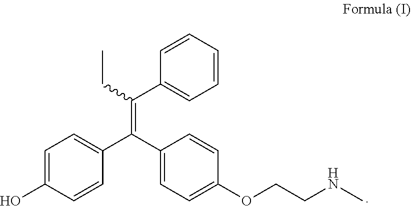 [SMILES: CCC(=C(c1ccc(O)cc1)c1ccc(OCCNC)cc1)c1ccccc1]

(Z)-endoxifen can be represented by Formula (II):

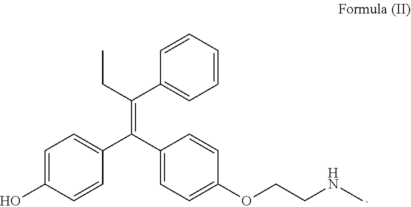 [SMILES: CC/C(=C(\c1ccc(O)cc1)c1ccc(OCCNC)cc1)c1ccccc1]

(E)-endoxifen can be represented by Formula (III):

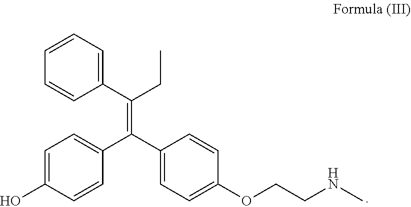 [SMILES: CC/C(=C(/c1ccc(O)cc1)c1ccc(OCCNC)cc1)c1ccccc1]

In some embodiments, the endoxifen compositions of the present disclosure may comprise (Z)-endoxifen as at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen (e.g., (Z)-endoxifen and (E)-endoxifen), by weight, in the composition. In some embodiments, the composition comprises at least 90% of (Z)-endoxifen with respect to total endoxifen, by weight, in the composition. In some embodiments, the composition comprises at least 95% of (Z)-endoxifen with respect to total endoxifen, by weight, in the composition. In some embodiments, the composition comprises at least about 96%, at least about 97%, at least about 98%, at least about 99%, at least about 99.5%, or at least about 99.9% of (Z)-endoxifen with respect to total endoxifen, by weight, in the composition. In some embodiments, the endoxifen compositions of the present disclosure may comprise (E)-endoxifen as no more than about 10%, no more than about 8%, no more than about 6%, no more than about 7%, no more than about 8%, no more than about 9%, no more than about 2%, or no more than about 1% of total endoxifen (e.g., (Z)-endoxifen and (E)-endoxifen), by weight, in the composition.

In some embodiments, the endoxifen compositions of the present disclosure may comprise a ratio of (Z)-endoxifen to (E)-endoxifen (Z:E ratio) that is at least about 50:50, at least about 60:40, at least about 64:36, at least about 70:30, at least about 80:30, at least about 82:18, at least about 85:15, at least about 90:10, at least about 94:6, at least about 95:5, at least about 96:4, at least about 97:3, at least about 98:2, at least about 99:1, or about 100:0. In some embodiments, the endoxifen compositions of the present disclosure may comprise a ratio of (Z)-endoxifen to (E)-endoxifen (Z:E ratio) that is from about 50:50 to about 100:0, from about 60:40 to about 100:0, from about 64:36 to about 100:0, from about 70:30 to about 100:0, from about 80:30 to about 100:0, from about 82:18 to about 100:0, from about 85:15 to about 100:0, from about 90:10 to about 100:0, from about 94:6 to about 100:0, from about 95:5 to about 100:0, from about 96:4 to about 100:0, from about 97:3 to about 100:0, from about 98:2 to about 100:0, or from about 99:1 to about 100:0.

An endoxifen composition of the present disclosure (e.g., for use in a combination therapy) may comprise an amount of (Z)-endoxifen formulated as a dosage form, such as a tablet or capsule. In some embodiments, a single endoxifen dosage form (e.g., a single capsule or a single tablet) may comprise about 1 mg, about 2 mg, about 4 mg, about 8 mg, or about 10 mg, of (Z)-endoxifen.

Endoxifen, also referred to as 4-hydroxy-N-desmethyl-tamoxifen, may include a polymorphic, salt, free base, co-crystal, or solvate form of endoxifen. Examples of salts of (Z)-endoxifen suitable for the endoxifen compositions of the present disclosure include pharmacologically acceptable salts such as salts with inorganic acids, salts with organic acids, salts with amino acids and the like. Examples of (Z)-endoxifen salts with inorganic acids include salts with hydrochloric acid, hydrobromic acid, nitric acid, sulfuric acid, phosphoric acid, and the like. Examples of salts with organic acids include salts with formic acid, acetic acid, trifluoroacetic acid, fumaric acid, oxalic acid, tartaric acid, maleic acid, methanesulfonic acid, benzenesulfonic acid, p-toluenesulfonic acid, and the like. Examples of the salts with acidic amino acids include salts with aspartic acid, glutamic acid, citric acid, and the like.

Examples of anion salts of (Z)-endoxifen include arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, and triethiodide. Examples of cation salts of (Z)-endoxifen selected from the group consisting of benzathine, clemizole, chloroprocaine, choline, diethylamine, diethanolamine, ethylenediamine, meglumine, piperazine, procaine, aluminum, barium, bismuth, lithium, magnesium, potassium, and zinc, and the like. In some embodiments, the present disclosure provides that embodiments include salts made with acids that are not pharmaceutically acceptable.

In some embodiments, an endoxifen composition of the present disclosure comprises salts of (Z)-endoxifen selected from the group consisting of acetate, arecoline, benzathine, benzoic, besylate, benzosulfonate, bicarbonate, bitartarate, butylbromide, citrate, camysylate, clemizole, chloroprocaine, choline, diethylamine, diethanolamine, ethylenediamine, formate, fumarate, glucolate, gluconate, glutamate, glycollylarsanilate, hexylresorcinate, hydrabamine, hydrobromide, hydrochloride, hydroxynapthanoate, isethionate, malate, maleate, mandelate, meglumine, mesylate, methylbromide, methylbromide, methylnitrate, methylsulfate, methanesulfonate, mucate, napsylate, nitric, nitrate, oxalate, pamaoate (Embonate), pantothenate, perchloric, phosphate, diphosphate, piperazine, procaine, polygalacuronate, p-toluenesulfonate, salicylate, stearate, succinate, sulfate, sulfonate, sulfuric, tannate, tartarate, teoclate, triethiodide, trifluoroacetate, aluminum, barium, bismuth, lithium, magnesium, potassium, and zinc aluminum, barium, bismuth, lithium, magnesium, potassium, and zinc, or any combination thereof. In some embodiments, the salt is (Z)-endoxifen gluconate. Endoxifen gluconate can be selected from the group consisting of (Z)-endoxifen D-gluconate, (Z)-endoxifen L-gluconate, or a combination thereof.

While the content of (Z)-endoxifen or a polymorph or a salt thereof in the endoxifen compositions of the present disclosure varies depending on the dosage form of the composition, target disease, severity of disease, and the like, it is an amount generally corresponding or equivalent to from about 0.01 mg to about 200 mg of (Z)-endoxifen. One of skill in the art will recognize that when a composition includes salts of (Z)-endoxifen, the endoxifen salt may be in an equivalent amount on the basis of (Z)-endoxifen to be released.

In some embodiments, the endoxifen may comprise one or more polymorphic forms, such as Form I, Form II, or Form III, of endoxifen. A polymorphic form may be distinguished by its x-ray powder diffraction pattern. In some embodiments, a method of treating a cancer may comprise administering a pharmaceutical composition comprising endoxifen predominantly as polymorph Form I. In some embodiments, the pharmaceutical composition comprises endoxifen predominantly as polymorph Form II. In some embodiments, the pharmaceutical composition comprises endoxifen predominantly as polymorph Form III.

In some embodiments, polymorphic Form I is characterized by an x-ray powder diffraction pattern comprising major peaks at 16.8±0.3°, 17.1±0.3° and 21.8±0.3° 2-theta. In some embodiments, polymorphic Form II is characterized by an x-ray powder diffraction pattern comprising major peaks at 7.0±0.3°, 11.9±0.3°, 14.0±0.3° and 18.4±0.3° 2-theta. In some embodiments, polymorphic Form III is characterized by an x-ray powder diffraction pattern comprising major peaks at 11.9±0.3°, 13.9±0.3°, 17.1±0.3° and 17.7±0.3° 2-theta.

The endoxifen in a composition of the present disclosure may be present in one or more polymorphic forms (e.g., one or more of Form I, Form II, Form III, or combinations thereof). For example, a composition of the present disclosure may comprise at least about 90% of total endoxifen, by weight, as polymorph Form I. In another example, a composition of the present disclosure may comprise at least about 95% of total endoxifen, by weight, as polymorph Form I. When a particular percentage by weight of endoxifen is a single polymorphic form (e.g., Form I, Form II, or Form III), the remainder of endoxifen in the composition may be some combination of amorphous endoxifen or one or more polymorphic forms of endoxifen excluding the single polymorphic form. In some embodiments, the endoxifen composition comprises (Z)-endoxifen predominantly as polymorph Form I. In some embodiments, the composition comprises (Z)-endoxifen predominantly as polymorph Form II. In some embodiments, the composition comprises (Z)-endoxifen predominantly as polymorph Form III. In certain embodiments, the (Z)-endoxifen can be any combination of Forms I, II and III.

In certain aspects, the present disclosure provides crystalline forms of endoxifen, including crystalline forms of (Z)-endoxifen free base and crystalline forms of mixtures of (E)-endoxifen and (Z)-endoxifen. The present disclosure further provides pharmaceutical compositions of endoxifen comprising the crystalline forms described herein. A crystalline form of endoxifen may provide the advantage of bioavailability and stability, suitable for use as an active ingredient in a pharmaceutical composition. Variations in the crystal structure of a pharmaceutical drug substance or active ingredient may affect the dissolution rate (which may affect bioavailability, etc.), manufacturability (such as ease of handling, ability to consistently prepare doses of known strength) and stability (such as thermal stability, shelf life, etc.) of a pharmaceutical drug product or active ingredient. Such variations may affect the preparation or formulation of pharmaceutical compositions in different dosage or delivery forms, such as solid oral dosage forms including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, process control, or combinations thereof. Thus, crystalline forms of endoxifen may provide advantages such as: improving the manufacturing process of an active agent or the stability or storability of a drug product form of the compound or an active ingredient, and/or having suitable bioavailability and/or stability as an active agent.

The use of certain solvents and fractional crystallization methods may produce different polymorphic forms of endoxifen, including any one or more of polymorphic Forms I, II and III, which may exhibit one or more favorable characteristics described above. In some embodiments, a polymorphic form (e.g., Form I, Form II, or Form III) may affect one or more properties of a composition comprising endoxifen. For example, a polymorphic form (e.g., Form I, Form II, or Form III) of a therapeutic agent (e.g., endoxifen) may affect one or more of the dissolution rate, the solubility, the absorption rate, the Cₘₐₓ, the AUC, the Tₘₐₓ, or the t_{1/2}, of the therapeutic agent in a composition of the present disclosure. In some embodiments, the polymorphic form of endoxifen may confer one or more properties that favorably contribute to manufacturability of a composition of the present disclosure. In some embodiments, the polymorphic form of endoxifen may confer improved stability to a composition of the present disclosure. The processes for the preparation of the polymorphs described herein, and characterization of these polymorphs are described in greater detail below.

### Polymorphic Form I

In certain aspects, the present disclosure provides compositions comprising polymorphic Form I of endoxifen. In some embodiments, at least 90% by weight of the endoxifen in the composition is the (Z)-isomer (i.e., (Z)-endoxifen). The term “substantially as shown in” when referring, for example, to an XRPD pattern, includes a pattern that is not necessarily identical to those depicted herein, but that falls within the limits of experimental error or deviations when considered by one of ordinary skill in the art. The relative intensities of XRPD peaks can vary, depending upon the particle size, the sample preparation technique, the sample mounting procedure, and the particular instrument employed. Moreover, instrument variation and other factors can affect the 2-theta (2θ) values. Accordingly, when a specified 2-theta angle is provided, it is to be understood that the specified 2-theta angle can vary by the specified value ±0.5°, such as ±0.4°, ±0.3°, ±0.2°, or ±0.1°. As used herein, “major peak” refers to an XRPD peak with a relative intensity greater than 30%, such as greater than 35%. Relative intensity is calculated as a ratio of the peak intensity of the peak of interest versus the peak intensity of the largest peak in the XRPD pattern.

Polymorphic Form I may be characterized by an x-ray powder diffraction pattern comprising major peaks at 16.8±0.3°, 17.1±0.3° and 21.8±0.3° 2-theta. In some embodiments, the x-ray powder diffraction pattern of polymorphic Form I may be further characterized by one or more peaks selected from 16.0±0.3°, 18.8±0.3° and 26.5±0.3° 2-theta. In some embodiments, the x-ray powder diffraction pattern of polymorphic Form I may be further characterized by one or more peaks selected from 12.3±0.3°, 28.0±0.3° and 29.0±0.3° 2-theta. In some embodiments, the x-ray powder diffraction pattern of polymorphic Form I may be further characterized by one or more peaks selected from 12.3±0.3°, 16.0±0.3°, 18.8±0.3°, 26.5±0.3°, 28.0±0.3° and 29.0±0.3° 2-theta.

In some embodiments, the present disclosure provides a composition comprising polymorphic Form I of endoxifen. Greater than 90%, 95% or 99% by weight of the endoxifen in the composition may be polymorphic Form I. In some embodiments, the composition comprises 0.01 mg to 200 mg of polymorphic Form I. In some embodiments, at least about 5%, at least about 10%, about at least 20%, at least about 25%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen, by weight, in a composition may be present as polymorphic Form I.

### Polymorphic Form II

In certain aspects, the present disclosure provides compositions comprising polymorphic Form II of endoxifen. In some embodiments, the composition comprises the (E)-isomer and the (Z)-isomer of endoxifen (i.e., (E)-endoxifen and (Z)-endoxifen) in an E/Z ratio between about 0.9 and about 1.3, such as about 1.1. Polymorphic Form II may be characterized by an x-ray powder diffraction pattern comprising major peaks at 7.0±0.3°, 11.9±0.3°, 14.0±0.3° and 18.4±0.3° 2-theta. In some embodiments, the x-ray powder diffraction pattern of polymorphic Form II may be further characterized by one or more peaks selected from 6.6±0.3°, 13.3±0.3° and 20.0±0.3° 2-theta. the x-ray powder diffraction pattern of polymorphic Form I may be further characterized by one or more peaks selected from 6.6±0.3°, 13.3±0.3°, 20.0±0.3° and 22.0±0.3° 2-theta.

In certain embodiments, the present disclosure provides a composition comprising polymorphic Form II. Greater than 90%, 95% or 99% by weight of endoxifen in the composition may be polymorphic Form II. In some embodiments, the composition comprises 0.01 mg to 200 mg of polymorphic Form II. In some embodiments, at least about 5%, at least about 10%, about at least 20%, at least about 25%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen, by weight, in a composition may be present as polymorphic Form II.

### Polymorphic Form III

In certain aspects, the present disclosure provides compositions comprising polymorphic Form III of endoxifen. In some embodiments, the composition comprises the (E)-isomer and the (Z)-isomer of the endoxifen (i.e., (E)-endoxifen and (Z)-endoxifen) in an E/Z ratio between about 0.9 and about 1.3, such as about 1.1. Polymorphic Form III may be characterized by an x-ray powder diffraction pattern comprising major peaks at 11.9 0.3°, 13.9±0.3°, 17.1±0.3° and 17.7±0.3° 2-theta. In some embodiments, the x-ray powder diffraction pattern of polymorphic Form II may be further characterized by one or more peaks selected from 18.2±0.3°, 22.5±0.3° and 26.8±0.3° 2-theta. In some embodiments, the x-ray powder diffraction pattern of polymorphic Form II may be further characterized by one or more peaks selected from 18.2±0.3°, 22.5±0.3°, 25.3±0.3° and 26.8±0.3° 2-theta.

In certain embodiments, the present disclosure provides a composition comprising polymorphic Form III. Greater than 90%, 95% or 99% by weight of the endoxifen in the composition may be polymorphic Form III. In some embodiments, the composition comprises 0.01 mg to 200 mg of polymorphic Form III. In some embodiments, at least about 5%, at least about 10%, about at least 20%, at least about 25%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen, by weight, in a composition may be present as polymorphic Form III.

### Pharmaceutical Compositions

A composition for treatment of ovarian cancer may be formulated for as a pharmaceutical composition. For example, a composition comprising endoxifen (e.g., (Z)-endoxifen) may be formulated as a pharmaceutical composition. In another example, a composition comprising a phosphoinositide 3-kinase inhibitor (e.g., alpelisib) may be formulated as a pharmaceutical composition. In some embodiments, a composition for treating ovarian cancer may comprise endoxifen (e.g., (Z)-endoxifen) and phosphoinositide 3-kinase inhibitor (e.g., alpelisib) formulated in a single pharmaceutical composition.

In various embodiments, a pharmaceutical composition provided herein comprises from about 1% to about 99.99%, about 5% to about 95%, about 5% to about 90%, about 10% to about 80%, about 15% to about 70%, about 20% to about 60%, from about 30% to about 95%, from about 50% to about 90%, from about 60% to about 90%, from about 60% to about 80%, or from about 70% to about 80% by weight of one or more excipients. In certain embodiments, the composition provided herein comprises about 99.99%, about 95%, about 90%, about 85%, about 80%, about 75%, about 70%, about 65%, about 60%, about 55%, or about 50% by weight of one or more excipients. In certain embodiments, the composition provided herein comprises about 99.99%, about 99%, about 98%, about 97%, about 96%, about 95%, about 94%, about 93%, about 92%, about 91%, about 90%, about 89%, about 88%, about 87%, about 86%, or about 85% by weight of one or more excipients. In certain embodiments, the composition provided herein comprises about 85%, about 84%, about 83%, about 82%, about 80%, about 79%, about 78%, about 77%, about 76%, about 75%, about 74%, about 73%, about 72%, about 71%, about 70%, about 69%, about 68%, about 67%, about 66%, or about 65% by weight of one or more excipients. In certain embodiments, the composition provided herein comprises about 55%, about 54%, about 53%, about 52%, about 51%, about 50%, about 49%, about 48%, about 47%, about 46%, or about 45% by weight of one or more excipients. In certain embodiments, the composition provided herein comprises about 30%, about 29%, about 28%, about 27%, about 26%, about 25%, about 24%, about 23%, about 22%, about 21%, or about 20% by weight of one or more excipients.

Examples of excipients that can be used in the compositions formulated for oral administration are provided herein and can include, but are not limited to, one or more of bulking agents, binders, fillers, disintegrating agents, lubricants, glidants, control release agents, enteric coatings, film-forming agents, plasticizers, colorants, sweeteners, flavoring agents and the like, or any combination thereof.

Binders suitable for use in the pharmaceutical compositions provided herein include, but are not limited to, sucrose, starches such as corn starch, potato starch, or starches such as starch paste, pregelatinized starch, and starch 1500, PEG 6000, METHOCEL™, WALOCEL™ HM, LUVITEC®, caprolactam, AVICEL®, SMCC, UNIPURE®, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL® PH 101, AVICEL® PH 103 AVICEL® RC 581, AVICEL® PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL® PH 103 and Starch 1500 LM.

Examples of fillers suitable for use in the pharmaceutical compositions provided herein include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose dextrates, kaolin, mannitol, silicic acid, sorbitol, starch, pregelatinized starch, and mixtures thereof.

One or more binder or filler in compositions is typically present in from about 10% to about 99% (wt/wt) of the composition or the dosage form. In some embodiments, binders and/or fillers in a composition comprise about 15% to 99%, about 20% to 60%, about 25% to 55%, about 30% to 50%, about 35% to 60%, about 50% to 99% (wt/wt) of the composition.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. In some embodiments, the disintegrant is deep in the oral solid dosage form to delay disintegration. The amount of disintegrant used varies based upon the type of formulation, and is readily discernible to those of ordinary skill in the art.

In some embodiments, compositions comprise from 0.5% to 15% (wt/wt) of disintegrant. In some embodiments, compositions comprise from 1% to 5% (wt/wt) of disintegrant in the composition. In another embodiment, the disintegrant is 1% to 25%, 2% to 20%, 5% to 15%, 8% to 12%, or about 10% (wt/wt) of the composition.

Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and mixtures thereof. Additional lubricants include, for example, a syloid silica gel (AEROSIL ° 200, manufactured by W.R. Grace Co. of Baltimore, Md.), a coagulated aerosol of synthetic silica (marketed by Degussa Co. of Plano, Tex.), CAB-O-SIL® (a pyrogenic silicon dioxide product sold by Cabot Co. of Boston, Mass.), Q7-9120 (Dow Corning), and mixtures thereof. If used at all, lubricants are typically used in an amount of less than 1% (wt/wt) of the compositions or dosage forms into which they are incorporated. In yet another embodiment, the lubricant is 0.1% to 3%, such as 0.5% to 1% (wt/wt), of the composition.

Plasticizers may be added to control the softness or pliability of oral dosage forms such as shell of a capsule, caplet, or a tablet and thus, may improve the mechanical properties of the pH-sensitive materials of the coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt/wt), of the composition.

Examples of glidants include, but are not limited to, colloidal silicone dioxide, cellulose, calcium phosphate, di or tri-basic and the like.

As an example of sweeteners or sweetening agents include sucrose, saccharin, dextrose, maltose, sugar substitutes, aspartame, xylitol, mannitol, cyclamate, sucralose, maltitol, sorbitol, acesulfame K and the like.

Examples of flavoring agents include peppermint, methyl salicylate, peppermint, spearmint, methyl salicylate, raspberry, red berry, strawberry, pineapple, orange, cherry, and the like.

Compositions formulated for oral delivery as disclosed herein, for example, tablets, caplets, and capsules, may be coated with one or more enteric coating agent, control release agent or film forming agent to control or delay disintegration and absorption of the compositions comprising endoxifen or salts thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di-tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, a pharmaceutical composition comprising endoxifen (e.g., (Z)-endoxifen) may be formulated as a sustained release composition. In some embodiments, a pharmaceutical composition comprising a phosphoinositide 3-kinase inhibitor (e.g., alpelisib) may be formulated as a sustained release composition. Sustained release agent present in a sustained release composition of the present disclosure may be any sustained release agent known in the art to slow the release of a hydrophobic drug such as (Z)-endoxifen or a polymorph or a salt thereof.

Examples of sustained release agents include cellulosic ethers, gums, acrylic resins such as polymers and copolymers of acrylic acid, methacrylic acid, methyl acrylate, methyl methylacrylate, and combinations thereof, polyvinyl pyrrolidine, and protein-derived compounds. Examples of cellulosic ethers include hydroxyalkyl celluloses, hydroxyethyl celluloses, hydroxypropyl celluloses, hydroxypropylmethyl celluloses (HPMC or hypromellose, for example Nos. 2208, 2906, 2910), carboxyalkyl celluloses, and carboxymethyl celluloses. In some embodiments, the at least one sustained release agent is a pH sustained release agent such as acid insoluble polymers which become increasingly soluble and permeable above pH 5.0 but remaining impermeable below pH 5.0. Such controlled release polymers target upper small intestines and/or colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers, including those available commercially from Evonik or Rohm ((EUDRAGIT® sustained release polymers EUDRAGIT® RL (high permeability), EUDRAGIT® RS (low permeability) and EUDRAGIT® NM 30D (low permeability)—alone or in any combination thereof to achieve the desired permeability for sustained release. The viscosity of sustained release agents may be any viscosity suitable for sustained release of (Z)-endoxifen or a polymorph or a salt thereof. In certain embodiments, the viscosity of the at least sustained release agent ranges from about 1000 mPa·s to about 150,000 mPa·s. In some embodiments, the sustained release delivery system includes one or more SR/release rate controlling agents with viscosity ranging from about 1000 mPa·s to about 10,000 mPa·s, from about 10,000 mPa·s to about 70,000 mPa·s, from about 70,000 mPa·s to about 150,000 mPa·s. or a combination thereof. In some embodiments, the present disclosure provides that the sustained release delivery system includes two or more sustained release agents. Each sustained release agent may have the same viscosity or a differing viscosity, for example one sustained release agent may have a viscosity ranging from about 1000 mPa·s to about 10,000 mPa·s, while other sustained release agent may have a viscosity of about 10,000 mPa·s to about 70,000 mPa·s or about 70,000 mPa·s to about 150,000 mPa·s.

In some embodiments, the sustained release agent is HPMC/hypromellose (e.g., Nos. 2208, 2906, 2910). Hypromellose to be used in the present disclosure has a weight molecular average of about 20,000-500,000. In some embodiments, hypromellose has a molecular weight average of generally 20,000-250,000. Hypromellose is commercially available from Dow Chemicals under the trade name METHOCELL™, for example, METHOCELL™ K100 (average molecular weight 26,000, 2% viscosity; 75,000-140,000 mPa.$); METHOCELL™ K15M (average molecular weight 120,000, 2% viscosity; 15,000 cP, 13275-24,780 mPa.$); METHOCELL™ K4M (average molecular weight 86,000, 2% viscosity; 4,000 cP, 75,000-140,000 mPa·s). Hypromellose of one grade may be used alone or in combination with another grade.

When a sustained release composition showing release of (Z)-endoxifen or a polymorph or a salt thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16, at least 18 hours, at least 24 hours, at least 48 hours, and at least 72 hours is obtained, in some embodiments the sustained release agent, such as Hypromellose, has an average molecular weight generally ranging from 15,000 to 140,000 Daltons. In at least one embodiment, the average molecular weight of about 15,000 Daltons.

The amount of sustained release agent in the composition may be any amount effective to delay the release of the therapeutic agent (Z)-endoxifen, or a polymorph or a salt thereof, for about 2 hours post-dose to protect the therapeutic agent from the acidic environment of the stomach and allow passage of the therapeutic agent through the stomach into the intestines and prolong such release for a period of about 2 hours to about 72 hours. The amount of sustained release agent in the composition may be any amount effective to provide a slower rate of release of the therapeutic agent (Z)-endoxifen, or a polymorph or a salt thereof as compared with the reference product. In some embodiments, the amount of sustained release agent in the composition may be any amount effective to delay the release of the therapeutic agent (Z)-endoxifen, or a polymorph or a salt thereof, for at least about 1 hour, at least about 1.1 hours, at least about 1.2 hours, at least about 1.3 hours, at least about 1.4 hours, at least about 1.5 hours, at least about 1.6 hours, at least about 1.7 hours, at least about 1.8 hours, at least about 1.9 hours, at least about 2 hours, at least about 2.1 hours, at least about 2.2 hours, at least about 2.3 hours, at least about 2.4 hours, or at least about 2.5 hours post-dose, as compared with the reference product.

When a sustained release composition shows percentage dissolution ranging from about 0% to 35% at 3 hours, from about 35% to about 55% at 12 hours, and from about 65% to 85% at 24 hours in a dissolution test according to the 75 RPM USP paddle method and using pH 1.2 at 37° C. for 2 hours in simulated gastric fluid and pH 6.8 at 37° C. for 24 hours in simulated intestinal fluid as a test medium, at least one sustained release agent, such as Hypromellose (HPMC), may generally be present in a sustained release composition of the present disclosure in amounts from about 0.1% to about 99%, from about 0.1% to about 90%, from about 5% to about 90%, from about 5% to about 80%, from about 5% to about 70%, and from about 5% to about 60% w/w of the sustained release composition. In some embodiments, the sustained release agent (e.g., a gum, an acrylic resin, methacrylic acid, methyl acrylate, methyl methylacrylate, polyvinyl pyrrolidine, a protein-derived compound, a hydroxyalkyl cellulose, a hydroxyethyl cellulose, a hydroxypropyl cellulose, a hydroxypropylmethyl celluloses, a carboxyalkyl cellulose, or, a carboxymethyl cellulose) may be present in an amount of from about 10% to about 40%, from about 10% to about 50%, from about 10% to about 60%, from about 20% to about 40%, from about 20% to about 50%, from about 20% to about 60%. In some embodiments, the sustained release agent may be present in an amount of at least about 10%, at least about 20%, at least about 30%, or at least about 40%. Such sustained release compositions of the present disclosure as disclosed herein release (Z)-endoxifen or polymorphs or salts thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16 hours, at least 18 hours, at least 24 hours, at least 48 hours, and at least 72 hours.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, NJ as a powder or a 30% aqueous dispersion; or under the tradename EASTACRYL®, from Eastman Chemical Co., Kingsport, TN, as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® S12,5, EUDRAGIT® FS 30D, EUDRAGIT® E100, EUDRAGIT® E 12,5, and EUDRAGIT® PO. In at least one embodiment, the composition comprises EUDRAGIT® L100-55. EUDRAGIT® RS and RL and EUDRAGIT® NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

For time delay or delayed-release pharmaceutical preparations of oral dosage forms, glyceryl monostearate, glyceryl distearate, and acid-insoluble polymers, for example polymethacrylate pH-sensitive polymer-based coatings can be used, (e.g., as coating material, i.e., enteric coating agents, for enteric coating of capsules, caplets, and tablets). Commercial sources for delayed-release oral dosage forms are available, for example DRCAPS® made of hypromellose (HPMC) from CAPSUGEL®, USA. Such delayed-release oral dosage forms are acid-resistant and can resist acidity as seen in stomach for at least 30 min, such as for at least 1 hour, for at least 1.5 hour, or for at least 2 hours. Such delayed release oral dosage forms can release at least 40%, at least 50%, at least 60%, at least 70%, at least 80% or at least 90% of the endoxifen or salts thereof in the intestines (small intestines, large intestine/colon, etc.).

In an aspect of the present disclosure, the enteric tablets, enteric caplets, and enteric capsules may be uncoated. Hard uncoated capsules with enteric capability using intrinsically enteric capsule technology (for example, ENTRINSIC™ Drug Delivery available from CAPSUGEL®) are suitable for the purpose of the present disclosure.

In various embodiments, the enteric tablet is a hard tablet made with free-flowing powder of (Z)-endoxifen or a salt thereof. In various embodiments, the enteric capsule is a capsule made with free-flowing powder of (Z)-endoxifen or a salt thereof. In various embodiments, the enteric tablet is a hard tablet made with free-flowing powder of endoxifen or a polymorph thereof. In various embodiments, the enteric capsule is a capsule made with free-flowing powder of endoxifen or a polymorph thereof.

In some embodiments, the enteric capsule is a non-animal-based capsule, such as a hypromellose capsule (for example, commercially available self-gelling VCAPS®, VCAPS® Plus, VCAPS® enteric, other enteric capsules made using XCELLODOSE®, ENCODE colonic delivery technology, and ENTRINSIC™ drug delivery technology from CAPSUGEL®). Other technologies known in the art and available commercially (for example, QUALICAPS®, USA, Nutrascience, USA, etc.) for the formulating enteric forms of oral solid dosage forms can also be utilized. In at least one embodiment, the capsule is an API-in-capsule, meaning that the (Z)-endoxifen free base or salts thereof is filled neat into the capsule. In such API-in-capsule oral dosage forms, the active ingredient, (Z)-endoxifen or salts thereof can be free flowing powders or micronized powders. When the dosage form is a capsule, in at least one embodiment, the capsule can be a seamless capsule or a banded capsule.

An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. An oral dosage form may be of any size suitable for oral administration, for example, size 0, size 2, and the like.

One of skill in the art will further recognize that compositions disclosed herein may comprise one or more of the excipients known in the art and disclosed herein in any combination appropriate for a desired formulation or preparation. Additional excipients may generally be found in Remington's The Science and Practice of Pharmacy, Meade Publishing Co., United States Pharmacopeia/National Formulary. One of skill in the art will be able to select suitable excipients necessary for the preparation of the formulations and appropriate dosage forms compatible with the route of administration based on his or her skill and knowledge in the art and the disclosures made herein. In all cases, the ultimate dosage form should be sterile and stable under the conditions of manufacture and storage.

For formulations of the solid dosage compositions disclosed herein, as the water activity (Aw) is less than 0.75, testing Total Aerobic Plate Count (TAC) and USP indicator organism is typically not necessary. The publication, “Microbial Bioburden on Oral Solid Dosage Form,” by Jose E. Martinez, Pharmaceutical Technology, February 2002, pages 58 to 70, is hereby incorporated by reference in its entirety.

For other formulations, such as liquid or fluid formulations with water activity of less than 0.75, Tests for Specified Microorganisms (S. aureus, Ps. aeruginosa, Salmonella, C. albicans, Clostridia, E. coli and Bile Tolerant Gram-negative bacteria) in compliance with USP Guidelines Chapter 62 may not need to be performed.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the terms “a,” “an,” and “the” include plural reference unless the context dictates otherwise.

It is specifically understood that any numerical value cited herein includes all values from the lower value to the upper value, i.e., all possible combination of numerical values between the lowest value and the highest value enumerated are to be considered to be expressly stated in this application and the endpoint of all ranges are included within the range and independently combinable. For example, if a concentration range or beneficial range is stated as 1% to 50%, it is intended that values such as 2% to 40%, 10% to 30%, or 1% to 3% etc., are expressly enumerated in this specification. It is also to be understood that if a concentration or dose is stated as a specific value such as 1 mg or 10 mg, it is intended that it is intended to include 10% variation. As another example, a stated concentration of 20% is intended to include values ±10%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

As used herein, the terms “active pharmaceutical ingredient”, “active ingredient”, “API,” “drug,” “active,” “actives” or “therapeutic agent” may be used interchangeably to refer to the pharmaceutically active compound(s) in a pharmaceutical composition. This is in contrast to other ingredients in the compositions, such as excipients, which are substantially or completely pharmaceutically inert. A suitable API in accordance with the present disclosure is one where there is or likely may be patient compliance issues for treating a certain disease, condition, or disorder. The therapeutic agent as used herein includes the active compound and its salts, prodrugs, and metabolites. As used herein the term “drug” means a compound intended for use in diagnosis, cure, mitigation, treatment, and/or prevention of disease in man or other animals.

As used herein, “adjuvant therapy” refers to a therapy that follows a primary therapy and that is administered to subjects at risk of relapsing. Adjuvant systemic therapy in case of breast cancer or reproductive tract cancer, for example with tamoxifen, usually begins soon after primary therapy to delay recurrence, prolong survival or cure a subject.

Embodiments that reference throughout this specification to “a compound”, such as compounds of Formula (I), Formula (II), Formula (III) and Formula (IV), include the polymorphic, salt, free base, co-crystal, and solvate forms of the formulas and/or compounds disclosed herein. Thus, the appearances of the phrases “a compound”, “compound of Formula (I)”, “compound of Formula (II)”, “compounds of Formula (III)” and “compound of Formula (IV)” include Form I of the compound of Formula (IV), Forms II-III of the compounds of Formula (III), the free base of the compound of Formula (IV), the free base of the compounds of Formula (III), and/or the gluconate salts as described herein.

The terms “crystalline form”, “polymorph” and “Form” may be used interchangeably herein and are meant to include all crystalline and amorphous forms of the compound, including, for example, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compound of the present disclosure include crystalline and amorphous forms of those compounds, including, for example, polymorphs, pseudopolymorphs, salts, solvates, hydrates, unsolvated polymorphs (including anhydrates), conformational polymorphs, and amorphous forms of the compounds, as well as mixtures thereof.

All compounds disclosed herein are further understood to include all possible isotopes of atoms occurring in the compounds. Isotopes include those atoms having the same atomic number but different mass numbers. By way of example, and without limitation, isotopes of hydrogen include tritium and deuterium and isotopes of carbon include ¹¹C, ¹³C and ¹⁴C.

As used herein and in the claims, the terms “comprising,” “containing,” and “including” are inclusive, open-ended and do not exclude additional unrecited elements, compositional components, or method steps. Accordingly, the terms “comprising” and “including” encompass the more restrictive terms “consisting of” and “consisting essentially of.”

As used herein, the term “combination therapy” refers to the use of a composition described herein in combination with one or more additional treatment. Treatment in combination therapy can be any treatment such as any prophylactic agent, therapeutic agent (such as chemotherapy), radiotherapy, surgery, and the like. The combination can refer to inclusion of a therapeutic or prophylactic agent in a same composition as a composition disclosed herein (for example, in the same capsule, tablet, ointment, etc.) or in separate compositions (for example, in 2 separate capsules). The separate compositions may be in a different dosage form. The use of the terms “combination therapy” and “in combination with” does not restrict the order in which a composition described herein and prophylactic and/or therapeutic agent and/or treatment are administered to a subject in need thereof. Compositions of the present disclosure can be administered prior to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year before), concomitant with, or subsequent to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year after) administration of one or more prophylactic and/or therapeutic agent and/or treatment to a subject in thereof. Combination therapy as used herein can also refer to treatment of a subject having a single disease or multiple diseases, for example, prostate cancer in men and gynecomastia.

As used herein, the term “pharmaceutically acceptable carrier” or “carrier” means a pharmaceutically acceptable material, composition, or vehicle, such as a liquid or solid filler, diluent, excipient, solvent, or encapsulating material, involved in carrying or transporting one or more of the compounds of the present disclosure from one tissue, organ, or portion of the body or across the skin.

As used herein, the term “pharmaceutically acceptable salt” refers to any salt (e.g., obtained by reaction with an acid or a base) of a compound of the present disclosure that is physiologically tolerated in a subject (e.g., a mammal, and/or in vivo, ex-vivo, in vitro cells, tissues, or organs). A “salt” of a compound of the present disclosure may be derived from inorganic or organic acids and bases. Suitable anion salts include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide.

Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

As used herein, the term “pharmaceutical composition” means a combination of the active agent (e.g., an active pharmaceutical compound or ingredient, API) with a carrier, inert or active (e.g., a phospholipid), making the compositions particularly suitable for diagnostic or therapeutic uses in vitro, in vivo, or ex vivo.

As used herein, the terms “subject,” “patient,” “participant,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

All methods described herein can be performed in a suitable order unless otherwise indicated or otherwise clearly contradicted by context. The use of any and all examples, or exemplary language (e.g., “such as” and “the like”) is intended merely to illustrate the invention and does not pose a limitation on the scope of the invention unless otherwise claimed. No language in the specification should be construed as any indicating any non-claimed element as essential to practice of the invention as used herein.

### EXAMPLES

The invention is further illustrated by the following non-limiting examples.

### Example 1

### 3D Tumor Organoid Screening of Ovarian Cancer Therapies

This example describes screening ovarian cancer therapies using three-dimensional tumor organoid cultures. Three-dimensional tumor organoid cultures were grown from tumor cells isolated from a human patient with recurrent ovarian cancer. Cancer drugs and combination therapies were screened for efficacy against the tumor cells. A combination therapy of endoxifen and alpelisib produced a robust tumor response.

### Example 2

### Use of a Combination Therapy of Endoxifen and Alpelisib to Treat Ovarian Cancer

This example describes the use of a combination therapy of endoxifen and alpelisib to treat recurrent ovarian cancer. An oral formulation of (Z)-endoxifen is administered to a human subject with recurrent ovarian cancer in combination with alpelisib. Alpelisib is formulated for oral administration. The subject is administered 4 mg of (Z)-endoxifen and 300 mg of alpelisib are administered to the subject daily until tumors shrink or unacceptable toxicity is observed. Following treatment with the combination of (Z)-endoxifen and alpelisib, the ovarian tumors of the subject are reduced or eliminated, and the tumor recurrence frequency is decreased.

### Example 3

### Stable Pharmaceutical Formulations for Treatment of Ovarian Cancer

This example describes stable pharmaceutical formulations for treatment of ovarian cancer. (Z)-endoxifen and alpelisib are formulated together in a single tablet. The tablet contains 4 mg of (Z)-endoxifen and 300 mg of alpelisib. The tablet is administered to patients with ovarian cancer to treat the ovarian cancer once daily for at least 28 days, or until tumors shrink or unacceptable toxicity is observed. The single tablet formulation shows improved stability upon administration to patients compared to administration of (Z)-endoxifen and alpelisib formulated as separate tablets. The improved stability results in more consistent and sustained dosing compared to the separate formulations. Additionally, patient compliance and adherence to dosing regimen is improved in patients taking the single tablet formulation as compared to patients taking (Z)-endoxifen and alpelisib formulated as separate tablets.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating ovarian cancer in a subject in need thereof, the method comprising administering to the subject a therapeutically effective amount of endoxifen and a phosphoinositide 3-kinase inhibitor, thereby treating the ovarian cancer.

2. The method of claim 1, wherein the endoxifen is (Z)-endoxifen.

3. The method of claim 1 or claim 2, wherein the phosphoinositide 3-kinase inhibitor is selected from the group consisting of alpelisib, idelalisib, copanlisib, duvelisib, umbralisib, taselisib, perifosine, buparlisib, dactolisib, and voxtalisib.

4. The method of claim 4, wherein the phosphoinositide 3-kinase inhibitor is alpelisib.

5. The method of any one of claims 1-4, wherein the endoxifen is administered orally, intravenously, intra-arterially, intra-ovarianly, vaginally, parenterally, topically, or transdermally.

6. The method of any one of claims 1-5, wherein the phosphoinositide 3-kinase inhibitor is administered orally, intravenously, intra-arterially, intra-ovarianly, vaginally, parenterally, topically, or transdermally.

7. The method of any one of claims 1-6, wherein the endoxifen, the phosphoinositide 3-kinase inhibitor, or both are administered orally.

8. The method of any one of claims 1-7, wherein the endoxifen is formulated as a sustained-release composition.

9. The method of any one of claims 1-7, wherein the endoxifen is formulated as a delayed-release composition.

10. The method of any one of claims 1-9, wherein the endoxifen is administered to the subject daily.

11. The method of any one of claims 1-10, wherein the endoxifen is administered to the subject at a dose of no less than 1 mg and no more than 160 mg per day.

12. The method of any one of claims 1-11, wherein the endoxifen is administered to the subject at a dose of no less than 1 mg and no more than 40 mg per day.

13. The method of any one of claims 1-12, wherein the endoxifen is administered to the subject at a dose of no less than 1 mg and no more than 10 mg per day.

14. The method of any one of claims 1-13, wherein the endoxifen is administered to the subject at a dose of no less than 2 mg and no more than 5 mg per day.

15. The method of any one of claims 1-14, wherein the phosphoinositide 3-kinase inhibitor is administered to the subject daily.

16. The method of any one of claims 1-15, wherein the phosphoinositide 3-kinase inhibitor is administered to the subject at a dose of no less than 10 mg and no more than 600 mg per day.

17. The method of any one of claims 1-16, wherein the phosphoinositide 3-kinase inhibitor is administered to the subject at a dose of no less than 50 mg and no more than 400 mg per day.

18. The method of any one of claims 1-17, wherein the phosphoinositide 3-kinase inhibitor is administered to the subject at a dose of no less than 150 mg and no more than 300 mg per day.

19. The method of any one of claims 1-18, wherein the endoxifen is administered 1, 2, 3, or 4 times per day.

20. The method of any one of claims 1-19, wherein the phosphoinositide 3-kinase inhibitor is administered 1, 2, 3, or 4 times per day.

21. The method of any one of claims 1-20, wherein the endoxifen and the phosphoinositide 3-kinase inhibitor are administered once per day.

22. The method of any one of claims 1-21, wherein the endoxifen, the phosphoinositide 3-kinase inhibitor, or both are administered for at least 7, at least 14, at least 21, or at least 28 days.

23. The method of any one of claims 1-22, wherein the endoxifen and the phosphoinositide 3-kinase inhibitor are administered as a single tablet.
